(19) United States
(12) Patent Application Publication
(10) Pub. No.: US 2022/0401507 A1
(43) Pub. Date: Dec. 22, 2022

(54) Marijuana Chew

(71) Applicant: Alan T. Yurich, Babylon, NY (US)

(72) Inventor: Alan T. Yurich, Babylon, NY (US)

(21) Appl. No.: 17/230,734

(22) Filed: Jun. 17, 2021

(51) Int. Cl. A61K 36/73; A61K 47/46; A61K 9/00

(52) U.S. Cl. CPC A61K 36/73; A61K 47/46; A61K 9/0056

(57) ABSTRACT

I invented * Marijuana Chew * for the benefit of peace throughout my body; physical, mental and spiritual which begat no pain but recreation (simple walking and bending). I Invented * Marijuana Chew * to help me get off of the severe medications that I was on from my tenure at the WTC since Sep. 11, 2001 and from my inherited illnesses thence, while serving my community.

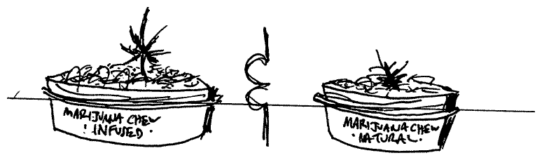

I began this invention slowly (under my state's license) and while it has morphed to an infused aspect now, incorporating my knowledge of certain herbs and spices and their traits and qualities in addition to the superb marijuana plant's dynamics, the flavors neutralize the pungency of the natural plant and fight inflammation; at least.

So, I bring you, “* Marijuana Chew * with natural.

While it's not the answer to everything. It can certainly allow you to breathe, move and live comfortably; enjoy responsibly.

I have invented * Marijuana Chew * against most people's thought of having to heat and or cook marijuana to get the full effect. As a World Trade Center First Responder. Sep. 11, 2001 and since. I have needed some serious medications throughout the years. When my state first opened up the access to Medicinal Marijuana, there were only oils under that license. These oils opened the doors and allowances to more of the benefits of the marijuana plant until providing access to the full bud. As a patient with breathing problems. I tasted the medicinal buds which then morphed into my consuming the plant (from the canister purchased through my state). The affect was surprising as the overall period of being pain free and relaxed throughout my body were overwhelmingly awesome for hours—without using heat. The lasting effects/affects STILL last for hours despite my tenure with this format of medicinal consumption—think about it, your eating everything that was seemingly made for such a purpose: recorded for centuries in Asia and the Middle East.

As an amateur cook and a holistic based person: since my first taste of the marijuana bud with my state license. I have created several flavorful concoctions for marijuana to marry within the canister of “Marijuana Chew”. Some of my flavors include rose, ginger, grapefruit and vanilla, etc. I have and am going to apply my knowledge as a professional patient and artist to cover all bases, to share with countless, faceless others, to allow for lawful recreational and medicinal folks—to share what has been on our planet before our conception, this “Marijuana Chew”.

While the naked flavor of marijuana can be quite pungent, the additives do marry well within my“Marijuana Chew”. I use what is commonly known and not so commonly known for anti-inflammatory purposes (Medicinally and holistically) and for satisfaction of taste & enjoyment. In any aspect, marijuana does have its natural powers, characteristics and qualities to relieve pain or stress & boredom and to increase the flow of imagination and depth to just about anything—where you choose, is up to you.

For myself as I experimented with my invention throughout the years that I've had the license to, it became important to be healthy: taste delicious: and for the sheer safety in every aspect of personal, physical and consumption. The package and overall flavor which is the ultimate melding of every trait and aspect listed above, is what I am patenting.

Please be responsible and adhere to local laws on every level while using this product.

There is No New Matter.

1. A Marijuana Chew comprising;
a dry marijuana bud being coarsely ground or flaky is the primary ingredient within its canister;
an additive ingredient is essential for an infused flavor, for example here, ginger;
a drop of water or one's saliva is essential in this invention as the melding ingredient [marijuana bud+ginger+saliva/water=Marijuana Chew infused];
place Marijuana Chew within your mouth as an edible benefit towards comfort. The invention claimed is something that I created if you will, while dealing with serious illnesses and the uses of several prescribed medications. The invention claimed is something that has helped make me personally medication free and mostly better feeling without severe side-effects.
